(19) United States
(12) Patent Application Publication
(10) Pub. No.: US 2019/0090970 A1
(43) Pub. Date: Mar. 28, 2019

(54) MANIPULATOR SYSTEM

(71) Applicant: OLYMPUS CORPORATION, Tokyo (JP)

(72) Inventor: Masao NICHOGI, Kouza-gun (JP)

(73) Assignee: OLYMPUS CORPORATION, Tokyo (JP)

(21) Appl. No.: 16/199,644

(22) Filed: Nov. 26, 2018

Related U.S. Application Data
(63) Continuation of application No. PCT/JP2016/066228, filed on Jun. 1, 2016.

(51) Int. Cl. A61B 34/37; A61B 34/20; A61B 34/00; B25J 3/00

(52) U.S. Cl. CPC A61B 34/37; A61B 34/20; A61B 2034/305; A61B 34/70; B25J 3/00; A61B 34/25

(57) ABSTRACT

A manipulator system includes a holding part, a distal joint part, an intermediate joint part, a proximal part, an operating part, and a controller. The distal joint part has a passive joint which bendably or rotatably couples the holding part and the intermediate joint part to each other, and a sensor which detects an orientation of the passive joint. The controller includes at least one processor. The at least one processor is configured to receive an operation command from the operating part; acquire an orientation of the passive joint from the sensor; and generate a command from the operation command to the motor on the basis of the acquired orientation of the passive joint and an orientation of the active joint.

This application is a continuation application based on PCT/JP2016/066228, filed on Jun. 1, 2016. The content of the PCT Application is incorporated herein by reference.

### BACKGROUND OF THE INVENTION

### Field of the Invention

The present invention relates to a manipulator system.

### Description of Related Art

Manipulator systems, which operate a medical device inserted into a body through a trocar by remote control, are known. When such manipulator systems are used, there is case where an insertion position of the medical device into the body is changed by attaching a plurality of the trocars to a body wall, extracting a medical device inserted into a certain trocar, and inserting the extracted medical device into another trocar. For example, PCT International Publication No. WO2013/018908 discloses a remote control type surgery system capable of flexibly responding to a change or the like in the insertion position of a medical device, such as forceps, into the body. According to the technique disclosed in PCT International Publication No. WO2013/018908, the insertion position of the medical device into the body can be easily changed by moving the medical device within a range of a movable zone of a multi-degree-of-freedom robot arm of the remote control type surgery system.

### SUMMARY OF THE INVENTION

An aspect of the invention is a manipulator system including: a holding part configured to hold a medical device; a distal joint part coupled to the holding part; an intermediate joint part coupled to the distal joint part, the intermediate joint part including an active joint; a proximal part coupled to the intermediate joint part; a motor coupled to the proximal part, the motor configured to generating power for operating the active joint; an operating part configured to be operated by an operator for operating the active joint; and a controller configured to control the motor. The distal joint part comprises: a passive joint which bendably or rotatably couples the holding part and the intermediate joint part to each other, and a sensor configured to detect an orientation of the passive joint. The controller includes at least one processor, the at least one processor is configured to: receive an operation command from the operating part; acquire the orientation of the passive joint from the sensor; and generate a command to the motor from the operation command on the basis of the orientation of the passive joint and an orientation of the active joint.

An aspect of the invention is a manipulator system including: a holding part configured to hold a medical device; a distal joint part coupled to the holding part; an intermediate joint part coupled to the distal joint part, the intermediate joint part including an active joint; a proximal part coupled to the intermediate joint part; a motor coupled to the proximal part, the motor is configured to generating power for operating the active joint; an operating part configured to be operated by an operator for operating the active joint; and a controller configured to control the motor. The distal joint part comprises: a passive joint configured to bendably or rotatably couple the holding part and the intermediate joint part to each other, the passive joint including a torque limiter, and a sensor configured to detect an orientation of the passive joint. The controller includes at least one processor, the at least one processor is configured to: receive an operation command from the operating part; acquire an orientation of the passive joint from the sensor; and generate a command of the motor from the operation command on the basis of the orientation of the passive joint and an orientation of the active joint.

An aspect of the invention is a method of controlling a manipulator system including: receiving a first command from an operating part for operating an active joint; detecting a first orientation of the active joint; detecting a second orientation of a passive joint; generating a second command of a motor for driving the active joint on the basis of the first command, the first orientation, and the second orientation; and transmitting the second command to the motor.

### BRIEF DESCRIPTION OF THE DRAWINGS

FIG. 1 is an overall view of a manipulator system of a first embodiment of the invention.

FIG. 2 is an enlarged view of a vicinity of a holding part of the manipulator system.

FIG. 3 is a block diagram of the manipulator system.

FIG. 4 is an explanatory view of the operation of the manipulator system.

FIG. 5 is an explanatory view of the operation of the manipulator system.

FIG. 6 is a block diagram of a manipulator system of a second embodiment of the invention.

FIG. 7 is an enlarged view of a vicinity of a holding part in a manipulator system of a third embodiment of the invention.

FIG. 8 is an enlarged view of a vicinity of a holding part in a manipulator system of a fourth embodiment of the invention.

### DETAILED DESCRIPTION OF THE INVENTION

### First Embodiment

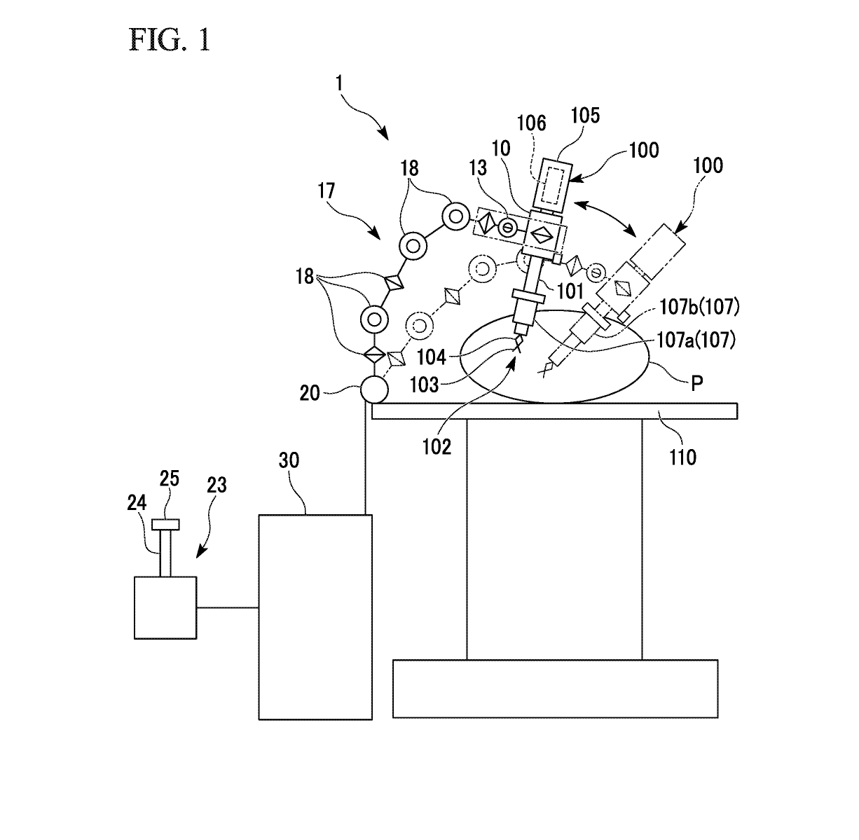

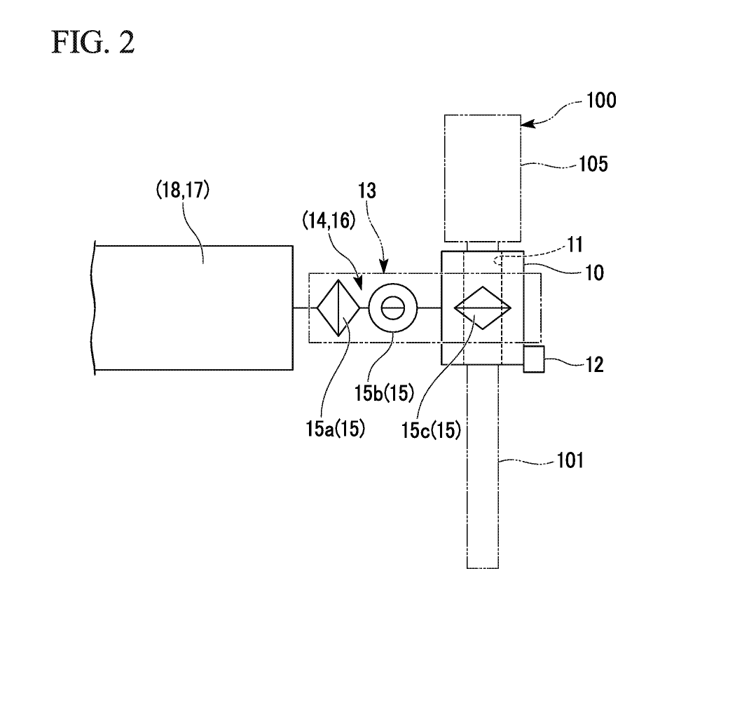

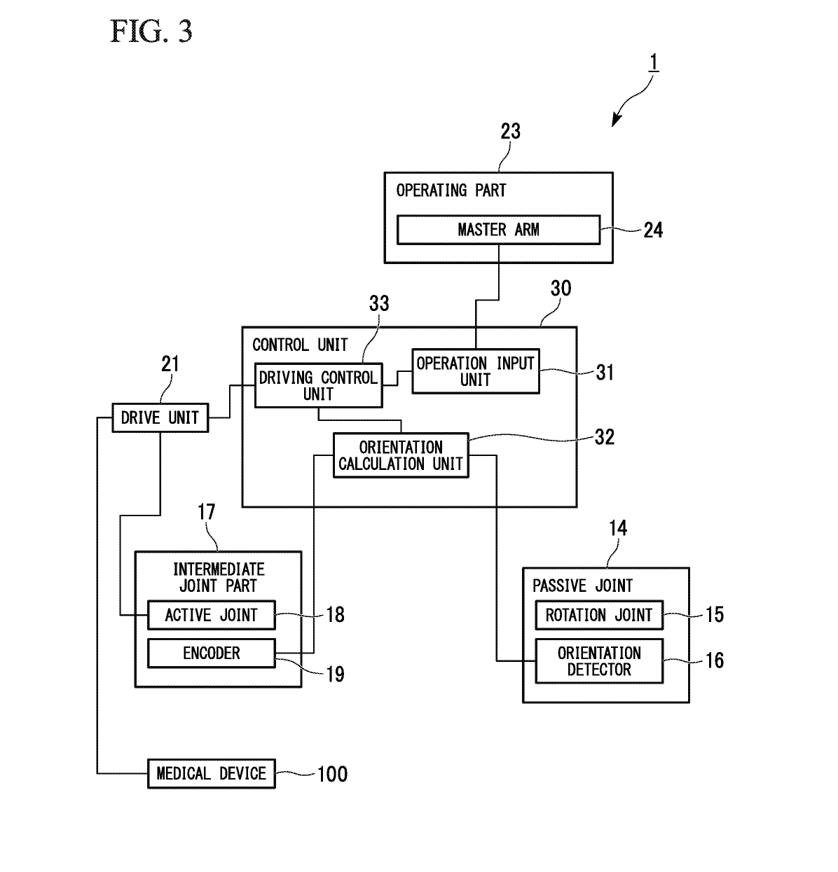

A first embodiment of the invention will be described. FIG. 1 is an overall view of the manipulator system of the present embodiment. FIG. 2 is an enlarged view of a vicinity of a holding part of the manipulator system. FIG. 3 is a block diagram of the manipulator system.

As shown in FIG. 1, a manipulator system 1 of the present embodiment is a system capable of attaching a medical device 100 to be inserted into the body of a patient P through a trocar 107. Examples of the medical device 100 to be used after being attached to the manipulator system 1 of the present embodiment are forceps, an incision tool, and the like to be used in laparoscopic surgery.

For example, the medical device 100 has an elongated shaft 101, a treatment part 102 arranged at a tip of the shaft 101, and a body part 105 arranged at a base end of the shaft 101. The treatment part 102 has, for example, a pair of jaws 103 capable of being opened and closed, and a joint 104 that swingably couples the treatment part 102 to the shaft 101.

Additionally, in the present embodiment, a power source 106 for operating the treatment part 102 is arranged inside the body part 105. The operation of the power source 106 within the body part 105 is controlled by a control unit 30 to be described below.

As shown in FIGS. 1 to 3, the manipulator system 1 includes a holding part 10, a distal joint part 13, an intermediate joint part 17, a proximal part 20, a drive unit (motor) 21, a control unit (controller) 30, and an operating part 23.

The holding part 10 holds the medical device 100. The holding part 10 of the present embodiment has a tubular part 11 into which the shaft 101 of the medical device 100, and a fixing mechanism 12 for clamping and fixing the shaft 101. The operation in which the fixing mechanism 12 fixes or releases the shaft 101 may be manual, or may be controlled by the control unit 30.

The distal joint part 13 is coupled to the holding part 10 and is coupled to the intermediate joint part 17. The distal joint part 13 has a passive joint 14 and an orientation detector (sensor) 16.

The passive joint 14 includes rotation joints 15 (a first joint 15a, a second joint 15b, a third joint 15c) having three axes, which are orthogonal to each other when the distal joint part 13 is in a linear state, as rotation centers, respectively.

The first joint 15a is a joint that is rotatable with respect to a tip joint (active joint 18) arranged at a tip of the intermediate joint part 17.

The second joint 15b is a joint having an axis, which extends in a direction orthogonal to the rotation center of the first joint 15a, as a rotation center.

The third joint 15c is a joint having an axis, which extends in a direction orthogonal to the rotation center of the second joint 15b, as a rotation center.

By providing the passive joint 14, the holding part 10 is swingable and rotatable with respect to the tip of the intermediate joint part 17.

The movement of the respective rotation joints 15 included in the passive joint 14 is limited such that the respective rotation joints 15 do not move due to the weight of the medical device 100. For example, the movement of the respective rotation joints 15 is limited by friction so that the respective rotation joints 15 do not easily move to such an extent that the weight of the medical device 100 can be supported. Additionally, the respective rotation joints 15 are fixed in a case where the medical device 100 is held, and each rotation joint 15 may have a clutch for making each rotation joint 15 freely movable in a case where an operator moves the medical device 100.

The orientation detector 16 is connected to the passive joint 14 and is connected to the control unit 30 (refer to FIG. 3).

The orientation detector 16 detects the orientations of the rotation joints 15 included in the passive joint 14. The orientation detector 16 includes, for example, a rotary encoder.

As shown in FIG. 1, the intermediate joint part 17 is coupled to the distal joint part 13.

The intermediate joint part 17 has a plurality of the active joints 18, and encoders 19 that detect the orientations of the respective active joints 18.

The plurality of active joints 18 are connected to the drive unit 21 by a power transmission member (not shown).

The encoders 19 detect the orientations of the plurality of active joints 18 provided in the intermediate joint part 17. The encoder 19 is connected to the control unit 30.

The proximal part 20 is coupled to the intermediate joint part 17.

The proximal part 20 is attached to a placement table 110 on which a patient who is a target of surgery using the manipulator system 1 of the present embodiment is placed. The proximal part 20 can be attached to any of a plurality of positions on the placement table 110. Additionally, the proximal part 20 may be coupled to the placement table 110 so as to be movable in a predetermined range with respect to the placement table 110.

The drive unit 21 is coupled to the proximal part 20. The drive unit 21 is connected to the active joints 18 via the power transmission member (not shown). The drive unit 21 is electrically connected to the control unit 30. The drive unit 21 generates the power for operating the active joints 18 in accordance with a command by the control unit 30.

In addition, instead of being coupled to the proximal part 20, the drive unit 21 may have an actuator (not shown) at each active joint 18 of the intermediate joint part 17 and output a command from the control unit 30 to the actuator of each active joint 18.

Additionally, the drive unit 21 of the present embodiment can transmit the power for operating the medical device 100 to the medical device 100 in accordance with the command from the control unit 30.

The operating part 23 is connected to the control unit 30. The operating part 23 has, for example, a master arm 24 for operating the active joints 18 and changing the position and orientation of the medical device 100. The master arm 24 has an input device 25 for performing an operation input for operating the treatment part 102 of the medical device 100.

As shown in FIGS. 1 and 3, the control unit 30 is electrically connected to the respective encoders 19 of the intermediate joint part 17, the orientation detector 16 of the distal joint part 13, the drive unit 21, the body part 105 of the medical device 100, and the operating part 23.

The control unit 30 is configured by at least one processor. At least one processor receives an operation command from the operating part, acquires an orientation of the passive joint from the sensor, and generates a command to the motor from the operation command on the basis of the acquired orientation of the passive joint and an orientation of the active joint.

The control unit 30 has an operation input unit 31 that receives an input from the operating part 23, an orientation calculation unit 32 that calculates the orientations of the holding part 10 and the medical device 100 on the basis of the orientations of the active joints 18 and the passive joint 14, and a driving control unit 33 that generates a command on the basis of the input to the operation input unit 31 and outputs the command to the drive unit 21.

The orientation calculation unit 32 acquires the orientations of the holding part 10 and the medical device 100 as coordinate data or the like in a coordinate system with a predetermined origin as a reference.

The driving control unit 33 generates a command including the movement direction and movement speed of the medical device 100 so as to correspond to the input to the operation input unit 31, with the coordinate data acquired by the orientation calculation unit 32 as a starting point, and outputs this command to the drive unit 21.

In the present embodiment, the orientation of the passive joint 14 is manually changed by the operator or the like, and the orientations of the active joints 18 are changed by the control unit 30 operating the drive unit 21 in accordance with an operation in the operating part 23. Here, the control unit 30 is capable of detecting the orientation of the passive joint 14 in the orientation detector 16, and is capable of recognizing the orientation of the passive joint 14 after change in a case where the operator or the like has changed the orientation of the passive joint 14. For this reason, even in a case where the operator or the like changes the orientation of the passive joint 14 or an external force is applied to the medical device 100 and the orientation of the passive joint 14 changes, the control unit 30 is capable of correctly recognizing the position and orientation of the medical device 100.

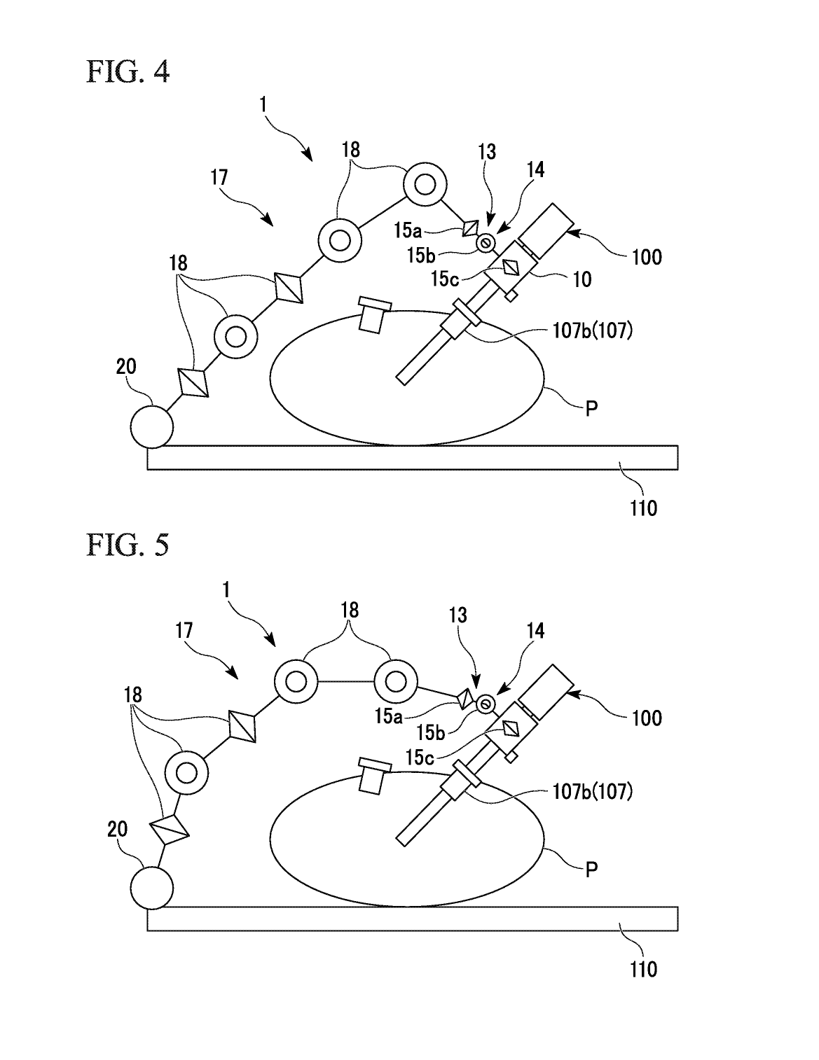

The action of the manipulator system 1 of the present embodiment will be described. FIGS. 4 and 5 are explanatory views of the operation of the manipulator system 1 of the present embodiment.

As shown in FIG. 1, when the manipulator system 1 of the present embodiment is used, the control unit 30 operates the drive unit 21 in order to change the position and orientation of the medical device 100. The medical device 100 is introduced into the body of the patient through the trocar 107 placed on a body wall of the patient. The indwelling position of the trocar 107 with respect to the body wall is determined in accordance with the contents of treatment for the patient. Additionally, there is a case where a plurality of the trocars 107 may be placed on the body wall.

If the medical device 100 is introduced into the body through the trocar 107, the control unit 30 of the manipulator system 1 recognizes the orientation of the medical device 100 in that state as an initial orientation, and sets the position of the trocar 107 as a fixed point. After the fixed point is set by the control unit 30, the control unit 30 operates the drive unit 21 on the basis of an operation for the operating part 23 such that the medical device 100 swings with the fixed point as a swinging center. Additionally, the control unit 30 can also operate the drive unit 21 so as to move the shaft 101 of the medical device 100 forward and backward in an axis direction thereof. In this case, the position of the fixed point in the medical device 100 changes in accordance with the forward and backward movement of the shaft 101.

In a case where the plurality of trocars 107 are placed in the body wall, there is a case where a trocar 107 into which the medical device 100 is to be inserted may be changed from a certain trocar 107 (for example, a first trocar 107a shown in FIG. 1) to another trocar 107 (for example, a second trocar 107b shown in FIG. 1). In this case, in the present embodiment, as the operator operates the passive joint 14 (refer to FIG. 2), the medical device 100 can be brought into an initial orientation suitable for treatment after the medical device 100 is moved to the vicinity of the other trocar 107b, and the respective active joints 18 in the intermediate joint part 17 can be brought into a state where the active joints 18 do not extremely bend.

FIGS. 4 and 5 illustrate examples of two different orientations in the manipulator system 1 holding the medical device 100 that is inserted into the trocar 107b and arranged at the suitable initial position. FIG. 4 illustrates the orientation of the manipulator system 1 in a case where the passive joint 14 is linear. FIG. 5 illustrates the orientation of the manipulator system 1 in a case where the second joint 15b among the rotation joints 15 constituting the passive joint 14 is operated, and the passive joint 14 is brought into a bending state. As compared to the orientation shown in FIG. 4, the orientation of the intermediate joint part 17 has a margin in the orientation shown in FIG. 5. Accordingly, if there is no passive joint 14, a movable zone of the intermediate joint part 17, and the orientation of the medical device 100 should be adjusted, whereas the movable zone of the intermediate joint part 17 can be widely left in the present embodiment.

As described above, the manipulator system 1 of the present embodiment can change the orientation of the medical device 100 without using the active joints 18 of the intermediate joint part 17 because the distal joint part 13 has the passive joint 14. As a result, in the manipulator system 1 of the present embodiment, the movable zone of the medical device 100 is wide.

### Second Embodiment

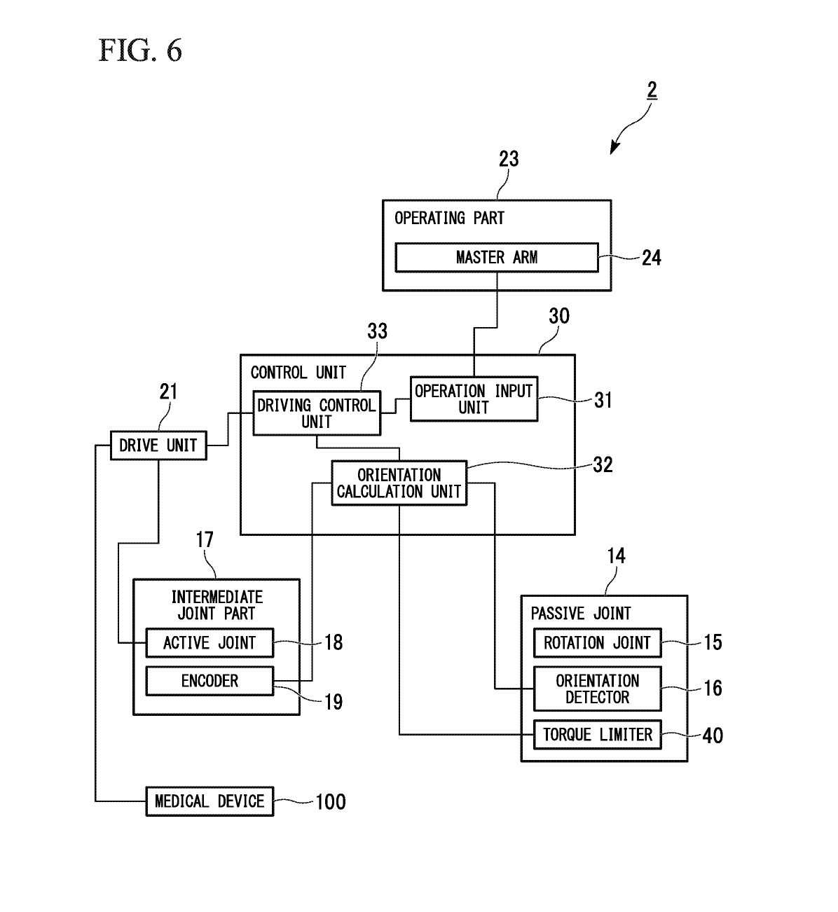

A second embodiment of the invention will be described. In addition, in the following respective embodiments, the same constituent elements as the constituent elements disclosed in the first embodiment will be designated by the same reference signs as those of the first embodiment, and the duplicate description thereof will be omitted. FIG. 6 is a block diagram of the manipulator system of the present embodiment.

As shown in FIG. 6, in a manipulator system 2 of the present embodiment, the passive joint 14 has a torque limiter 40. Additionally, the control unit 30 of the manipulator system 2 of the present embodiment is capable of detecting the presence or absence of the operation of the torque limiter 40 in the orientation calculation unit 32, and is capable of stopping the operation of the active joints 18 based on the operation to the operating part 23 in a case where the torque limiter 40 is detected to have been operated.

In the present embodiment, in a case where an external force is applied to the passive joint 14 to such an extent that the torque limiter 40 is operated, the orientation of the passive joint 14 changes in accordance with the external force. For example, in a case where the medical device 100 collides against another instrument or the like when the drive unit 21 has moved the medical device 100, the torque limiter 40 is operated in accordance with an external force that the medical device 100 receives from the instrument or the like. Then, the orientation of the passive joint 14 changes, and the operation of the active joint 18 stops.

### Third Embodiment

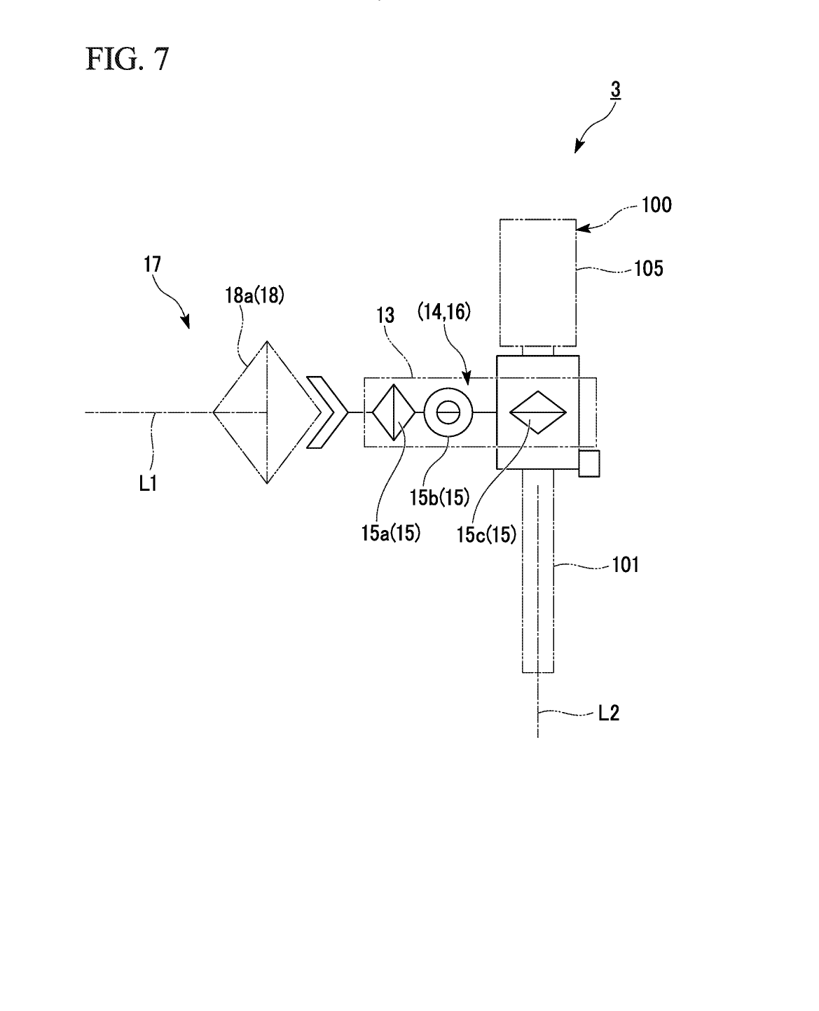

A third embodiment of the invention will be described. FIG. 7 is an enlarged view of a vicinity of a holding part of a manipulator system of the present embodiment.

As shown in FIG. 7, the intermediate joint part 17 in a manipulator system 3 of the present embodiment is connected to the distal joint part 13 by a roll joint 18a because an active joint 18 closest to a distal side the intermediate joint part 17 is the roll joint 18a.

Additionally, the holding part 10 is capable of holding the medical device 100 such that a longitudinal direction L2 of the medical device 100 is a direction orthogonal to a direction L1 in which a rotation center axis of the roll joint 18a extends.

In the present embodiment, since the body part 105 of the medical device 100 does not easily interfere with the intermediate joint part 17, a wide movable range of the intermediate joint part 17 can be secured.

In addition, in the present embodiment, the longitudinal direction L2 of the medical device 100 may not be orthogonal to the direction L1 in which the rotation center axis of the roll joint 18a extends. For example, the longitudinal direction of the medical device 100 may be a direction that intersects the direction, in which the rotation center axis of the roll joint 18a extends, at a predetermined angle.

### Fourth Embodiment

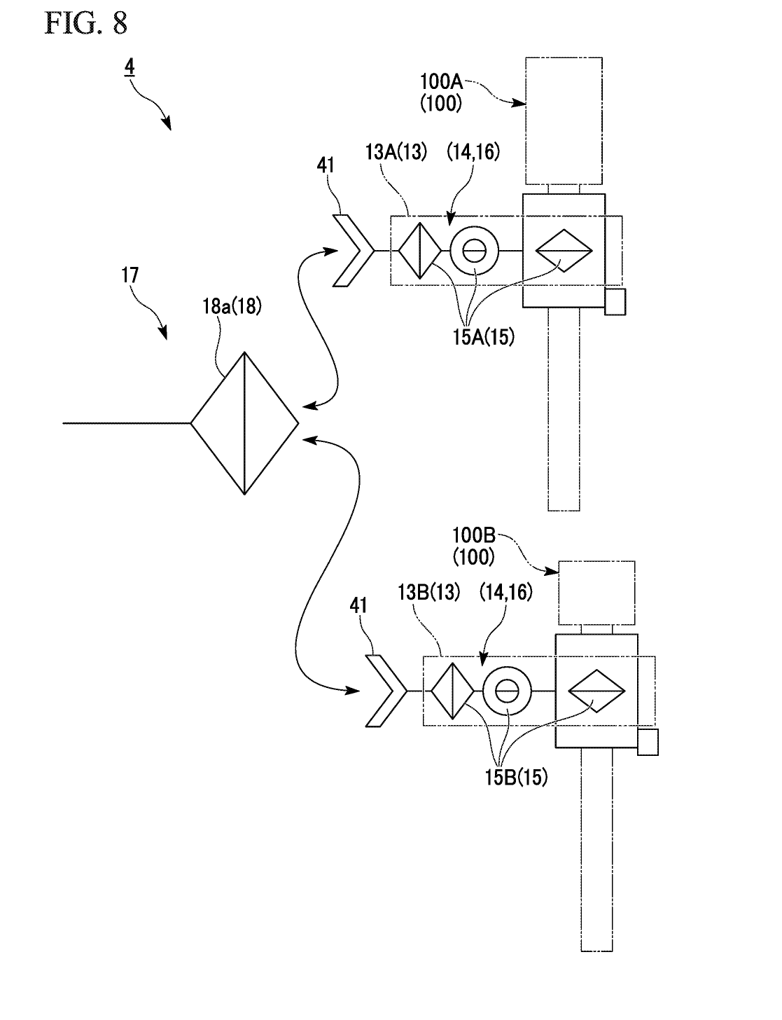

A fourth embodiment of the invention will be described. FIG. 8 is an enlarged view of a vicinity of a holding part of a manipulator system of the present embodiment.

As shown in FIG. 8, the distal joint part 13 of a manipulator system 4 of the present embodiment has an attaching and detaching part 41 that detachably attaches the distal joint part 13 and the intermediate joint part 17 to each other.

By providing the attaching and detaching part 41, in the present embodiment, the distal joint part 13 can be replaced with the intermediate joint part 17. In the present embodiment, the orientation detector 16 and the control unit 30 (refer to FIG. 3) can be attached and detached by contact or non-contact communication device.

For example, in the present embodiment, distal joint parts 13 (for example, a first distal joint part 13A and a second distal joint part 13B shown in FIG. 8) corresponding to the dimensions or the like of the medical device 100 can be attached to the intermediate joint part 17.

Additionally, in the present embodiment, the distal joint parts 13 in which the operation limitation states (for example, the magnitude of friction) of the respective rotation joints 15 are set in correspondence with the weight of the medical device 100 can be attached to the intermediate joint part 17. For example, a rotation joint 15A of the distal joint part 13A corresponding to a heavy medical device 100A can be set to have a greater friction than a rotation joint 15B of the distal joint part 13B corresponding to a light medical device 100B.

Additionally, in the present embodiment, in order to set a device on the distal side including the attaching and detaching part 41 as a device in a clean region and set a device on a proximal side including the intermediate joint part 17 as a device in an unclean region, a drape can be sandwiched between the attaching and detaching part 41 and the intermediate joint part 17.

According to the manipulator system 4 of the present embodiment, the width of selection of the medical device 100 capable of being attached to the manipulator system 4 can be expanded by having the distal joint parts 13 corresponding to the configuration of the medical device 100.

Although the embodiments of the invention have been described above in detail with reference to the drawings, specific configuration is not limited to these embodiments, and design changes are also included without departing from the scope of the invention. Additionally, the constituent elements shown in the above-described respective embodiments can be suitably configured in combination.

While preferred embodiments of the invention have been described and shown above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A manipulator system comprising:
a holding part configured to hold a medical device;
a distal joint part coupled to the holding part;
an intermediate joint part coupled to the distal joint part, the intermediate joint part including an active joint;
a proximal part coupled to the intermediate joint part;
a motor coupled to the proximal part, the motor configured to generating power for operating the active joint;
an operating part configured to be operated by an operator for operating the active joint; and
a controller configured to control the motor,
wherein the distal joint part comprises:
a passive joint which bendably or rotatably couples the holding part and the intermediate joint part to each other, and
a sensor configured to detect an orientation of the passive joint,
wherein the controller includes at least one processor, the at least one processor is configured to:
receive an operation command from the operating part;
acquire the orientation of the passive joint from the sensor; and
generate a command to the motor from the operation command on the basis of the orientation of the passive joint and an orientation of the active joint.

2. The manipulator system according to claim 1,
wherein the passive joint further comprises a torque limiter.

3. The manipulator system according to claim 2,
wherein the at least one processor is configured to:
detect an operation of the torque limiter,
generate a command for limiting the operation of the active joint in a case where the torque limiter is operated, and
transmit the command to the motor.

4. The manipulator system according to claim 1,
wherein the holding part is configured to hold the medical device such that a longitudinal direction of the medical device becomes a direction orthogonal to a rotation center axis direction of the active joint.

5. The manipulator system according to claim 1,
wherein the distal joint part further comprises an attaching and detaching part configured to detachably attach the distal joint part and the intermediate joint part to each other.

6. A manipulator system comprising:
a holding part configured to hold a medical device;
a distal joint part coupled to the holding part;
an intermediate joint part coupled to the distal joint part, the intermediate joint part including an active joint;
a proximal part coupled to the intermediate joint part;
a motor coupled to the proximal part, the motor is configured to generating power for operating the active joint;
an operating part configured to be operated by an operator for operating the active joint; and
a controller configured to control the motor,
wherein the distal joint part comprises:
a passive joint configured to bendably or rotatably couple the holding part and the intermediate joint part to each other, the passive joint including a torque limiter, and
a sensor configured to detect an orientation of the passive joint,
wherein the controller includes at least one processor, the at least one processor is configured to:
receive an operation command from the operating part;
acquire an orientation of the passive joint from the sensor; and
generate a command of the motor from the operation command on the basis of the orientation of the passive joint and an orientation of the active joint.

7. The manipulator system according to claim 6,
wherein the at least one processor is configured to:
detect an operation of the torque limiter,
generate a command for limiting the operation of the active joint in a case where the torque limiter is operated, and
transmit the command to the motor.

8. The manipulator system according to claim 6,
wherein the holding part is configured to hold the medical device such that a longitudinal direction of the medical device becomes a direction orthogonal to a rotation center axis direction of the active joint.

9. The manipulator system according to claim 6,
wherein the distal joint part further comprises an attaching and detaching part configured to detachably attach the distal joint part and the intermediate joint part to each other.

10. A method of controlling a manipulator system, comprising:
receiving a first command from an operating part for operating an active joint;
detecting a first orientation of the active joint;
detecting a second orientation of a passive joint;
generating a second command of a motor for driving the active joint on the basis of the first command, the first orientation, and the second orientation; and
transmitting the second command to the motor.

11. The method of controlling a manipulator system according to claim 10, further comprising:
detecting an operation of a torque limiter provided to the passive joint;
creating a third command for limiting an operation of the active joint when the torque limiter is operated; and
transmitting the third command to the motor.
